(19) United States
(12) Patent Application Publication
(10) Pub. No.: US 2026/0232715 A1
(43) Pub. Date: Aug. 13, 2026

(54) TREATMENT OF PANCREATIC CANCER

(71) Applicant: CAN-FITE BIOPHARMA LTD., Petah Tikva (IL)

(72) Inventors: Pnina FISHMAN, Herzliya (IL); Inbal ITZHAK, Kfar Saba (IL)

(21) Appl. No.: 19/150,095

(22) PCT Filed: Jan. 21, 2024
(86) PCT No.: PCT/IL2024/050085

§ 371 Date: Jul. 22, 2025

(30) Foreign Application Priority Data
Jan. 23, 2023 (IL) 300132

(51) Int. Cl. A61K 31/7076; A61K 31/4745; A61K 31/513; A61K 31/519; A61K 31/555; A61K 45/06; A61P 35/00

(52) U.S. Cl. CPC A61K 31/7076; A61K 31/4745; A61K 31/513; A61K 31/519; A61K 31/555; A61K 45/06; A61P 35/00

(57) ABSTRACT

The invention provides pharmaceutical compositions and methods of treating pancreatic cancer comprising administering an A₃ adenosine receptor (A₃AR) ligand, specifically namodenoson.

### FIELD OF THE INVENTION

This invention relates to the treatment of pancreatic cancer comprising administration of an A3AR ligand.

### REFERENCES

1) Ducreux M., et al., Semin. Oncol. 2019, 46, 28-38.
2) Stemmer S. M., et al. Oncologist. 2013; 18:25-26.
3) Stemmer, S. M., et al., Cancers 2021, 13, 187.
4) Vincenzi F., et al., Biomolecules 2023, 13, 1387.
5) WO 07/089507.

### BACKGROUND OF THE INVENTION

Pancreatic adenocarcinoma is a major cause of cancer-related death in Western countries, with an overall 5-year survival rate of 8%. One of the causes for this low survival rate is the absence of early detection methods. As a result, the tumor is not found until advanced stages with high likelihood of early metastasis.

Specific treatment depends on the size and location of the tumor, and whether it has metastasized. The most effective treatment is surgical removal of the cancerous part of the pancreas or the whole pancreas (termed pancreatectomy), but 5-year survival rates after surgical resection alone are low. Other treatments include radiation therapy, immunotherapy, or adjuvant therapy with gemcitabine or fluoropyrimidine (fluorouracil plus leucovorin) which has been shown to significantly improve outcomes and is recognized as standard care in patients with resected pancreatic cancer. Another adjuvant therapy is the combination of fluorouracil, leucovorin, irinotecan, and oxaliplatin (FOLFIRINOX).

However, recurrence rates remain high despite adjuvant treatment, with 69 to 75% of patients having a relapse within 2 years, as the efficacy of gemcitabine alone or in combination with other chemotherapies is modest, and checkpoint inhibitors failed to show a significant clinical benefit [Ducreux et al., 2019].

The A₃ adenosine receptor (A₃AR) is one of four receptors that mediate extracellular adenosine signaling [Vincenzi et al., 2023]. Its mRNA and protein expression levels are upregulated in different tumor cell types but not in the adjacent normal tissues.

Namodenoson (CF102, CI-IB-MECA) a synthetic ribose-based purine nucleoside is a selective orally bioavailable A₃AR agonist.

Phase I and II studies of namodenoson in advanced hepatocellular carcinoma (HCC) demonstrated excellent safety and efficacy in a subset of HCC patients with a Child-Pugh-B score of 7 (CPB7) [Stemmer et al. 2013; Stemmer et al., 2021]. A pivotal phase III trial investigating namodenoson in HCC CPB7 patients is ongoing.

### SUMMARY OF THE INVENTION

In a first of its aspects, the present invention provides a method of treating pancreatic cancer, said method comprising administering to a mammalian subject in need thereof an A₃ adenosine receptor (A₃AR) ligand or a pharmaceutical composition comprising said A₃AR ligand.

In another aspect, the present invention provides a method of increasing overall survival of subjects with pancreatic cancer, said method comprises administering an A₃AR ligand to said subject.

In another aspect, the present invention provides a pharmaceutical composition comprising an A₃AR ligand, and a pharmaceutically acceptable carrier or diluent wherein said pharmaceutical composition is for treating a pancreatic cancer in a mammalian subject.

In an embodiment said pancreatic cancer is an advanced pancreatic cancer.

In an embodiment, said A₃AR ligand is an A₃AR agonist or an A₃AR allosteric modulator.

In an embodiment, said A₃AR agonist is selected from the group consisting of N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (CI-IB-MECA, namodenoson).

In an embodiment, said A₃AR agonist is 2-chloro-N⁶-(3-iodobenzyl)-adenosine-5′-N-methyluronamide (CI-IB-MECA, namodenoson).

In an embodiment, said A₃AR allosteric modulator is selected from the group consisting of:
N-(3,4-Dichloro-phenyl)-2-cyclopentyl-1H-imidazo[4,5-c]quinolin-4-amine;
N-(3,4-Dichloro-phenyl)-2-cycloheptyl-1H-imidazo[4,5-c]quinolin-4-amine;
N-(3,4-Dichloro-phenyl)-2-cyclobutyl-1H-imidazo[4,5-c]quinolin-4-amine; and
N-(3,4-Dichloro-phenyl)-2-cyclohexyl-1H-imidazo[4,5-c]quinolin-4-amine.

In an embodiment, said method further comprises administration of an additional therapeutic agent.

In an embodiment, said additional therapeutic agent is a chemotherapeutic agent or an immunotherapeutic agent.

In an embodiment, said chemotherapeutic agent is selected from a group consisting of nucleoside chemotherapeutics, fluorouracil plus leucovorin, and fluorouracil plus leucovorin plus irinotecan plus oxaliplatin (known as FOLFIRINOX).

In an embodiment, said immunotherapeutic agent is an anti-tumor antibody and/or a checkpoint inhibitor.

In an embodiment, said pharmaceutical composition and said additional therapeutic agent are administered simultaneously.

In an embodiment, said pharmaceutical composition and said additional therapeutic agent are administered sequentially.

In an embodiment, said method further comprises irradiation of the tumor.

In an embodiment, said administration is performed prior to and/or after removal of the tumor.

In an embodiment, said A₃AR ligand is administered once daily, twice daily, or thrice daily.

In an embodiment, said A₃AR ligand is administered every 12 hours throughout the treatment period.

In an embodiment, said A₃AR ligand is administered in a continuous manner.

In an embodiment, said treatment period is divided into cycles (e.g., 4-week cycles).

In an embodiment, said mammalian subject is a human subject.

In an embodiment, said A₃AR ligand is administered at an amount of 50 μg/kg-10 mg/kg body weight, preferably 100 μg/kg-5 mg/Kg body weight, or 200 μg/kg-1 mg/Kg body weight.

In an embodiment, said A₃AR ligand is CI-IB-MECA and wherein said CI-IB-MECA is administered orally in a dose of 1-50 mg, preferably 5-30 mg twice daily.

In an embodiment, said subject receives the A₃AR ligand as a second-line therapy.

In an embodiment, said administration is for a treatment period of at least 9 months, at least 10 months, at least one year, at least 2 years, at least 3 years, at least 4 years or at least 5 years.

In another aspect, the present invention provides a kit comprising:
a pharmaceutical composition comprising an A₃AR ligand according to the invention; and
instructions for administration of the pharmaceutical composition for the treatment of a subject with pancreatic cancer.

### BRIEF DESCRIPTION OF THE DRAWINGS

To understand the invention and to see how it may be carried out in practice, a preferred embodiment will now be described, by way of non-limiting example only, with reference to the accompanying drawings, in which:

FIG. 1A is a graph showing the percentage of growth inhibition of BxPC-3 cells by various doses of namodenoson: 1 nM (67.4%±1.7), 0.1 nM (53.7%±6.3), and 0.01 nM (27.9%±2.3). ** p<0.001, *p<0.005.

FIG. 1B is a graph showing percentage of growth inhibition of BxPC-3 cells by various doses of namodenoson. Each datapoint represents an average of 6 independent experiments. The error bars represent SE. * p<0.001 (t-test vs. control).

FIG. 1C is a graph showing percentage of growth inhibition of BxPC-3 cells by 20 nM namodenoson in the presence or absence of the A₃AR antagonist MRS1523 (20 nM), as compared with control untreated cells. Each datapoint represents an average of 3 independent experiments. The error bars represent SE. ** p<0.01 (t-test vs. control).

FIG. 2 is a graph showing tumor size (mm²) in nude mice treated with Namodenoson for 35 days (from Day 22 to Day 57 post tumor inoculation). Each datapoint represents an average of 10 mice and error bars represent the corresponding SE.

FIGS. 3A-3D show Western blot analysis results of BxPC-3 cells treated with namodenoson vs. controls. (FIG. 3A) cell growth regulatory proteins downstream of A₃AR; (FIG. 3B) Wnt/β-catenin signaling pathway proteins; (FIG. 3C) RAS downstream proteins; and (FIG. 3D) apoptotic proteins.

### DETAILED DESCRIPTION OF EXEMPLARY EMBODIMENTS

The present invention is based on the surprising finding that namodenoson (CI-IB-MECA) effectively inhibits the growth of the pancreatic adenocarcinoma cell line BxPC-3.

As shown in the Examples, BxPC-3 pancreatic cells were cultured with namodenoson (5-20 nM for 24 hours at 37° C.), and the Presto Blue assay was used to monitor cell growth. Western blot analyses were performed on the BxPC-3 cells (20 nM namodenoson for 24 hours at 37° C.) to evaluate the expression levels of cell growth regulatory proteins. In vivo studies involved the subcutaneous inoculation of BxPC-3 cells into nude mice, randomizing the mice into namodenoson (10 μg/kg twice daily for 35 days) vs. control, and monitoring tumor size twice weekly. Treatment with namodenoson was associated with the significant dose-dependent inhibition of BxPC-3 cell growth, which was mitigated by the A₃AR antagonist MRS1523. Western blot analyses showed that namodenoson treatment modulated the expression of NF-κB, as well as proteins in the Wnt/β-catenin and the RAS signaling pathways, leading to the upregulation of apoptotic proteins (Bad, Bax). In vivo studies also showed the significant inhibition of pancreatic carcinoma tumor growth with namodenoson.

The present invention thus provides namodenoson as a treatment for pancreatic cancer.

The invention is described in the following detailed description with reference to therapeutic methods for the treatment of pancreatic cancer involving administration of an A₃AR ligand to a subject in need of same. The therapeutic methods and compositions of the invention may be applicable to the treatment of advanced pancreatic cancer, including cases where other treatment options were exhausted.

As used in the specification and claims, the forms “a”, “an” and “the” include singular as well as plural references unless the context clearly dictates otherwise. For example, the term “an A₃AR ligand” includes one or more ligands.

As used in the specification and claims, the forms “a”, “an” and “the” include singular as well as plural references unless the context clearly dictates otherwise. For example, the term “an A₃AR ligand” includes one or more ligands.

Further, as used herein, the term “comprising” is intended to mean that the method or composition includes the recited elements but does not exclude others. Similarly, “consisting essentially of” is used to define methods and compositions that include the recited elements but exclude other elements that may have an essential significant therapeutic activity towards pancreatic cancer. For example, a composition consisting essentially of an A₃AR ligand will not include or include only insignificant amounts (amounts that will have an insignificant effect on pancreatic cancer) of other active ingredients that have such an activity. Also, a composition consisting essentially of the A₃AR ligand as defined herein would not exclude trace contaminants from the isolation and purification method, pharmaceutically acceptable carriers, such as phosphate buffered saline, excipients, preservatives, and the like. “Consisting of” shall mean excluding more than trace elements of other elements. Embodiments defined by each of these transition terms are within the scope of this invention.

Further, all numerical values, e.g., concentration or dose or ranges thereof, are approximations which are varied (+) or (−) by up to 20%, at times by up to 10% of the stated values. It is to be understood, even if not always explicitly stated that all numerical designations are preceded by the term “about”. It also is to be understood, although not always explicitly stated, that the reagents described herein are merely exemplary and that equivalents of such are known.

There is provided by the present invention a pharmaceutical composition comprising an A₃ adenosine receptor (A₃AR) ligand and a pharmaceutically acceptable carrier or diluent for use in the treatment of pancreatic cancer in a mammalian subject.

A “pharmaceutical composition” in the context of the invention is intended to mean a combination of the active agent(s), together or separately, with a pharmaceutically acceptable carrier as well as other additives. The carrier may at times have the effect of improving the delivery or penetration of the active ingredient to the target tissue, improving the stability of the drug, slowing clearance rates, imparting slow-release properties, reducing undesired side effects etc. The carrier may also be a substance that stabilizes the formulation (e.g., a preservative). For examples of carriers, stabilizers, and adjuvants, see E. W. Martin, REMINGTON'S PHARMACEUTICAL SCIENCES, Mack Pub Co (June 1990).

As used herein, the term “an A₃ adenosine receptor (A₃AR) ligand” encompasses A₃AR agonists as well as A₃AR allosteric modulators.

A₃AR agonists are known in the art and are readily available. Generally, an A₃AR agonist is any compound that is capable of specifically binding to the adenosine A₃ receptor (“A₃R”), thereby fully or partially activating said receptor thereby yielding a therapeutic effect (e.g., a growth inhibitory or cytopathic effect). The A₃AR agonist is thus a molecule that exerts its prime effect through the binding and activation of the A₃AR. This means that at the doses it is being administered it essentially binds to and activates only the A₃R.

In an embodiment, the A₃AR agonist has a binding affinity (Kᵢ) to the human A₃AR of less than 1000 nM, desirably less than 500 nM, advantageously less 200 nM and even less than 100 nM, typically less than 50 nM, preferably less than 20 nM, more preferably less than 10 nM and ideally less than 5 nM. The lower the Kᵢ, the lower the dose of the A₃AR agonist (that may be used) that will be effective in activating the A₃R and thus achieving a therapeutic effect.

It should be noted that some A₃AR agonists can also interact with and activate other receptors with lower affinities (namely a higher Kᵢ). A molecule will be considered an A₃AR agonist in the context of the invention (namely a molecule that exerts its prime effect through the binding and activation A₃R) if its affinity to the A₃R is at least 3 times (i.e., its Kᵢ to the A₃R is at least 3 times lower), preferably 10 times, desirably 20 times and most preferably at least 50 times larger than the affinity to any other of the adenosine receptors.

The affinity of A₃AR agonists to the human A₃R as well as its relative affinity to the other human adenosine receptors can be determined by various assays, such as a binding assay. Examples of binding assays include providing membranes or cells having the receptor and measuring the ability of the A₃AR agonist to displace a bound radioactive agonist; utilizing cells that display the respective human adenosine receptor and measuring, in a functional assay, the ability of the A₃AR agonist to activate or deactivate downstream signaling events such as the effect on adenylate cyclase measured through increase or decrease of the cAMP level; etc. Clearly, if the administered level of an A₃AR agonist is increased such that its blood level reaches a level approaching that of the Kᵢ of the other adenosine receptors, activation of these receptors may occur following such administration, in addition to activation of the A₃R. An A₃AR agonist is thus preferably administered at a dose such that the blood level that will be attained will give rise to essentially only A₃R activation.

The characteristics of some adenosine A₃AR agonists and methods of their preparation are described in detail in, inter alia, U.S. Pat. Nos. 5,688,774; 5,773,423; 5,573,772; 5,443,836; 6,048,865; WO 95/02604; WO 99/20284; WO 99/06053; WO 97/27173 and WO 01/19360, all of which are incorporated herein by reference.

A specific group of A₃AR agonists are the N⁶-benzyladenosine-5′-uronamide derivatives. Some preferred N⁶-benzyladenosine-5′-uronamide derivatives are N⁶-2-(4-aminophenyl)ethyladenosine (APNEA), N⁶-(4-amino-3-iodobenzyl) adenosine-5′-(N-methyluronamide) (AB-MECA) and 1-deoxy-1-{6-[({3-iodophenyl}methyl)amino]-9H-purine-9-yl}-N-methyl-β-D-ribofuranuronamide (IB-MECA) and 2-chloro-N⁶-(3-iodobenzyl) adenosine-5′-N-methlyuronamide (CI-IB-MECA).

In a specific embodiment, the A₃AR agonist of the invention is 2-chloro-N⁶-(3-iodobenzyl) adenosine-5′-N-methlyuronamide (CI-IB-MECA), also known as namodenoson or CF-102.

When referring to an “A₃AR allosteric modulator” or “A₃ARM” it is to be understood as referring to the positive regulation, activation or increase of the receptor activity by binding of the allosteric modulator at the receptor's allosteric site which may be different from the binding site of the endogenous ligand or agonist thereof.

In one example, “modulation” denotes an effect of the A₃AR ligand on the receptor exhibited by an increase of at least 15% in the efficacy of the A₃ adenosine receptor by binding of the compound to the allosteric site of the receptor and/or by a decrease in dissociation rate of adenosine or an A₃AR agonist to the orthosteric binding site.

In one example, the modulation is by an A₃AR allosteric modulator (A₃ARAM) that is an imidazoquinoline derivative.

Specific imidazoquinoline derivatives which can be used as allosteric modulators of the A₃AR are listed below:
N-(3,4-Dichloro-phenyl)-2-cyclopentyl-1H-imidazo[4,5-c]quinolin-4-amine;
N-(3,4-Dichloro-phenyl)-2-cycloheptyl-1H-imidazo[4,5-c]quinolin-4-amine;
N-(3,4-Dichloro-phenyl)-2-cyclobutyl-1H-imidazo[4,5-c]quinolin-4-amine; and
N-(3,4-Dichloro-phenyl)-2-cyclohexyl-1H-imidazo[4,5-c]quinolin-4-amine.

The above imidazoquinoline derivatives are regarded as allosteric modulators as they were shown to have, on the one hand, reduced affinity, if any, to the orthosteric binding sites of the A₁ and A_{2A}, A_{2B} adenosine receptors and reduced affinity to the orthosteric binding site of the A₃ adenosine receptor, and on the other hand, high affinity to the allosteric site of the A₃ adenosine receptor [International Patent Application No. WO07/089507, incorporated herein by reference].

A specifically preferred imidazoquinoline derivative in accordance with the present disclosure is N-(3,4-Dichloro-phenyl)-2-cyclohexyl-1H-imidazo[4,5-c]quinolin-4-amine (also referred to at times by the abbreviation LUF6000 or CF602), being an A₃AR allosteric modulator.

The present disclosure also makes use of physiologically acceptable salts of an A₃AR selective ligand, such as the above-described compounds. A “physiologically acceptable salt” refers to any non-toxic alkali metal, alkaline earth metal, and ammonium salt commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the ligand with a suitable organic or inorganic acid. The acid addition salts are those which retain the biological effectiveness and qualitative properties of the free bases, and which are not toxic or otherwise undesirable. Examples include, inter alia, acids derived from mineral acids, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g., p-toluenesulphonic, acids.

The term “pharmaceutically acceptable carrier” in the context of the present invention denotes any one of inert, non-toxic materials, which do not react with the A₃AR agonist, and which can be added to formulations as diluents, carriers or to give form or consistency to the formulation.

In the context of the present invention the term “treatment” comprises treating pancreatic cancer to reverse, attenuate, or ameliorate disease symptoms. Thus, treatment refers to administering a therapeutically effective amount of an A₃AR ligand to achieve a desired therapeutic effect. The desired therapeutic effect may include, without being limited thereto, reduction in tumor volume, prevention of metastasis, and/or an increase in the patient's survival rate after surgical resection of the tumor.

The terms “pancreatic tumor” and “pancreatic cancer” are used interchangeably herein and refer to a proliferative disease of pancreatic cells. These terms encompass pre-cancerous and cancerous pancreatic cells, and particularly refer to pancreatic adenocarcinoma. The term also encompasses advanced pancreatic cancer, namely pancreatic cancer that spread from the original tissue, as well as recurrence of the cancer after treatment, including cases where other treatment options were exhausted, or an inoperable pancreatic cancer.

The A₃AR ligand can be administered in a single dose (one time medication) or as a continuous treatment, for a period of days, weeks, months or even years.

Further in the context of some embodiments of the present disclosure, the treatment A₃AR ligand is administered as a chronic treatment, for example long term (e.g., daily) administration at times even without an envisaged end point for the treatment, throughout the patient's life.

The composition of the present invention is administered and dosed in accordance with good medical practice, taking into consideration the clinical condition of the individual patient, the site and method of administration, scheduling of administration, patient age, sex, body weight and other factors known to medical practitioners. The choice of carrier will be determined in part by the specific active ingredient, as well as by the specific method used to administer the composition. Accordingly, there is a wide variety of suitable pharmaceutical compositions of the present invention.

The composition of the invention can be administered to the subject by a variety of delivery modes as known in the art, e.g., by oral, intraperitoneal, subcutaneous, transcutaneous, topical, intramuscular, intraarticular, subconjunctival, intranasal, or intraocular administration. In a preferred embodiment, the composition is administered orally. The carrier will be selected based on the desired form of the formulation.

The A₃AR ligand is administered in amounts which are sufficient to achieve a therapeutic effect, for example an anti-cancer effect. As will be appreciated, the amount of the A₃AR ligand will depend on the severity of the disease, the intended therapeutic regimen, and the desired therapeutic dose. By way of example, where the dose is 1 mg per day and the desired administration regimen is once daily administration, the amount of the A₃AR ligand in a pharmaceutical composition comprising same will be 1 mg. Where it is intended to divide this daily dose into 2 daily administrations, the amount of the active agent in the pharmaceutical composition will be 0.5 mg.

An amount effective to achieve the desired effect is determined by considerations known in the art. An “effective amount” for purposes herein must be effective to achieve a therapeutic effect, the therapeutic effect being as defined hereinbefore.

It is appreciated that the effective amount depends on a variety of factors including the affinity of the chosen A₃AR agonist to the A₃AR, its distribution profile within the body, a variety of pharmacological parameters such as half-life in the body, on undesired side effects, if any, on factors such as age and gender of the subject to be treated, etc. The effective amount is typically tested in clinical studies having the aim of finding the effective dose range, the maximal tolerated dose, and the optimal dose. The manner of conducting such clinical studies is well known to a person versed in the art of clinical development.

An amount may also at times be determined based on amounts shown to be effective in animals. It is well known that an amount administered to animals (e.g., mice) can be converted to an equivalent amount in another species (notably humans) using one of possible conversion equations well known in the art. Examples of conversion equations are as follows:

### Conversion I

| Species | Body Wt. (Kg) | Body Surf. Area (m²) | Km Factor |
|---|---|---|---|
| Mouse | 0.02 | 0.0066 | 3.0 |
| Rat | 0.15 | 0.025 | 5.9 |
| Human Child | 20.0 | 0.80 | 25 |
| Adult | 70.0 | 1.60 | 37 |

Body Surface area dependent Dose conversion: Rat (20 g) to Man (70 Kg) is 1/7 the rat dose. This means that in the present case 0.001-0.4 mg/Kg in rats equals to about 0.14-56 microgram/Kg in humans; assuming an average weight of 70 Kg, this would translate into an absolute dosage of about 0.01 to about 4 mg.

### Conversion II

The following conversion factors: Mouse=3, Rat=67. Multiply the conversion factor by the animal weight to go from mg/Kg to mg/m² for human dose equivalent.

| Species | Weight (Kg) | BSA (m²) |
|---|---|---|
| Human | 70.00 | 1.710 |
| Mouse | 0.02 | 0.007 |
| Rat | 0.15 | 0.025 |
| Dog | 8.00 | 0.448 |

According to this equation the amounts equivalent to 0.001-0.4 mg/Kg in rats for humans are 0.16-64 mg/Kg; namely an absolute dose for a human weighing about 70 Kg of about 0.011 to about 4.4 mg, like the range indicated in Conversion I.

In accordance with one embodiment of the invention, the administration of the A₃AR agonist is preferably by daily administration, between once and a few times a day, preferably once or twice a day, the dose in each administration being in the range of between about 1 to about 1000 μg/kg body weight, preferably less than 400 μg/kg body weight, and even less than 200 μg/kg body weight. Typically, the dose of A₃AR agonist is in a range of 1 to 100 g/kg body weight.

In an embodiment, the injection is administered in an extended-release formulation.

The therapeutic use of an A₃AR agonist may at times be in combination with other drugs or therapeutic procedures such as removal of the tumor (or the whole pancreas), irradiation, immunotherapy, and/or chemotherapy. Immunotherapy may include the administration of an anti-tumor antibody and/or a checkpoint inhibitor. Chemotherapy may include any drug, or drug combination, which are typically used for treating pancreatic cancer. Non-limiting examples include nucleoside chemotherapeutics (e.g., gemcitabine), fluoropyrimidine (fluorouracil plus leucovorin), or fluorouracil, plus leucovorin plus irinotecan plus oxaliplatin (known as FOLFIRINOX).

In such a combination treatment the chemotherapeutic drug and the A₃AR agonist may be given to patients at the same time or at different times, depending on the dosing schedule of each of the drugs.

The effective combination of the A₃AR agonist with an additional chemotherapeutic agent allows to reduce the dose of the A₃AR agonist and yet maintain therapeutic efficacy.

The invention has been described in an illustrative manner, and it is to be understood that the terminology which has been used is intended to be a description rather than a limitation. Obviously, many modifications and variations of the present invention are possible in view of the above teaching. It is therefore to be understood that within the scope of the appended claims, the invention may be practiced otherwise than as specifically described hereinafter.

### EXEMPLARY EMBODIMENTS

### Materials & Methods

### Reagents

Dulbecco's phosphate-buffered saline (PBS), RPMI medium, fetal bovine serum (FBS), RIPA buffer, and protease and phosphatase inhibitor cocktail (×100) were purchased from Thermo Fisher Scientific (Waltham, MA, USA). Dimethyl sulfoxide (DMSO) and MRS1523 were purchased from Sigma Chemical Co. (Rehovot, Israel). MRS1523 was dissolved in DMSO to yield a stock solution of 10 mM. Penicillin-streptomycin solution (x100) was purchased from IMBH (Beit Haemek, Israel).

Rabbit polyclonal antibodies against the phosphorylated-PKB/Akt (p-PKB/Akt), NF-κB, β-Catenin, A₃AR, PI3K, GSK-3β, cyclin D1, ERK 1/2, MEK 1/2, Raf, Bad, and Bax were purchased from Santa Cruz Biotechnology Inc. (Dallas, TX, USA).

Namodenoson (2-chloro-N⁶-(3-iodobenzyl)-adenosine-5′-N-methyl-uronamide), lot No. 1884-050-05, was prepared by WuXi (Wuxi, China), and was stored at 4° C. in the dark. Namodenoson stock solution (10 mM) was prepared daily by dissolving the namodenoson powder in DMSO. For the in vitro studies, this solution was further diluted to a final concentration of 5, 10, and 20 nM in RPMI medium; for the in vivo studies, the namodenoson stock solution was diluted in PBS.

Cell culture:

The BxPC-3 cell line (ATCC Cultures, Manassas, VA, USA) a cell line exhibiting epithelial morphology which was isolated from the pancreas tissue of a 61-year-old, female patient with adenocarcinoma was used for all the analyses. Cells (15×10⁴/mL) were maintained in RPMI medium supplemented with 10% fetal bovine serum (FBS), 200 mM glutamine, 100 U/ml penicillin, and 100 mg/ml streptomycin solution at 37° C. in a 5% CO₂ incubator. Cells were transferred to a freshly prepared medium twice weekly.

### In Vitro Assays

For the tumor cell growth experiments, BxPC-3 cells (15×10⁴/mL) were incubated in the culture medium with control, 5, 10, or 20 nM namodenoson in 96-well microtiter plates for 24 h at 37° C. in a 5% CO₂ incubator, after which cell growth inhibition was determined using the Presto Blue assay (Thermo Fisher Scientific, Waltham, MA, USA) used as per the manufacturer's instructions. Experiments under the same conditions with 20 nM namodenoson were also conducted with and without 20 nM of the A₃AR antagonist MRS1523 (diluted to the final concentration in RPMI medium).

Western blot analyses were performed using the same culture conditions as the Presto Blue experiments in 10 cm plates with 20 nM namodenoson or control. After incubation for 24 h at 37° C. in a 5% CO₂ incubator, the cell samples were rinsed with ice-cold PBS and transferred to ice-cold RIPA buffer with 1× protease and phosphatase inhibitor cocktail for 20 min. Cell debris was removed using centrifugation at 4° C. for 10 min, at 7500×g. The supernatant was utilized for the Western blot analyses. Protein concentrations were determined using the NanoDrop assay (ThermoFisher Scientific, Waltham, MA, USA). Equal amounts of the sample (50 μg) were separated by SDS-PAGE, using 12% membranes (Schleicher & Schuell, Keene, NH, USA). Membranes were blocked with 1% bovine serum albumin and incubated with the relevant primary antibody (dilution 1:1000) for 24 h at 4° C. Blots were then washed and incubated with the secondary antibody for 1 h at room temperature. Bands were recorded using BCIP/NBT color development kit (Promega, Madison, WI, USA).

3H-thymidine incorporation assay:

3H-thymidine incorporation assay was used to evaluate cell growth. BxPC-3 cells (5,000 cells/well) were incubated (37° C.) with CF102 (namodenoson) in different concentrations: 0.01 nM, 0.1 nM and 1 nM in a 96-well plate for 48 h. Each well was pulsed with 1 μCi 3H-thymidine for the last 24 h. The cells were harvested, and the 3H-thymidine uptake was determined in an LKB liquid scintillation counter (LKB).

### In Vivo Assays

The in vivo experiments were performed in accordance with the guidelines established by the Institutional Animal Care and Use Committee at Can-Fite BioPharma (Petah Tikva, Israel).

Male nude Balb/C mice (Harlan Laboratories, Jerusalem, Israel), aged 2 months (mean weight, 25 g), received a subcutaneous flank injection of BxPC-3 cells (2.5×10⁶). The mice were maintained on a standardized pelleted diet and supplied with tap water. At Day 22 (a tumor size of 150-200 mm³), the animals were randomly assigned to two groups each containing 10 animals (namodenoson, 100 μg/kg body weight given orally twice daily for 35 days, or control). Tumor width (W) and length (L) were measured twice weekly with a caliber, and tumor size was calculated (W²×L/2).

### Statistical Analysis

The inhibition/growth rate (vs. control) was calculated. All data are expressed as

mean±standard error (SE). Analyses were performed with t-test and p-value<0.05 was considered statistically significant. The statistical analyses were performed using Excel (Microsoft 365).

### Example 1: Namodenoson Inhibited Tumor Growth In Vitro in an A₃AR-Mediated Manner

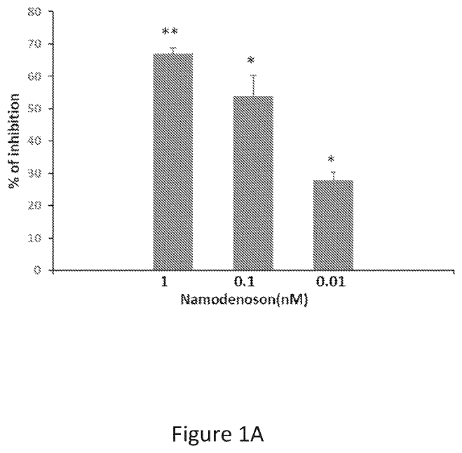

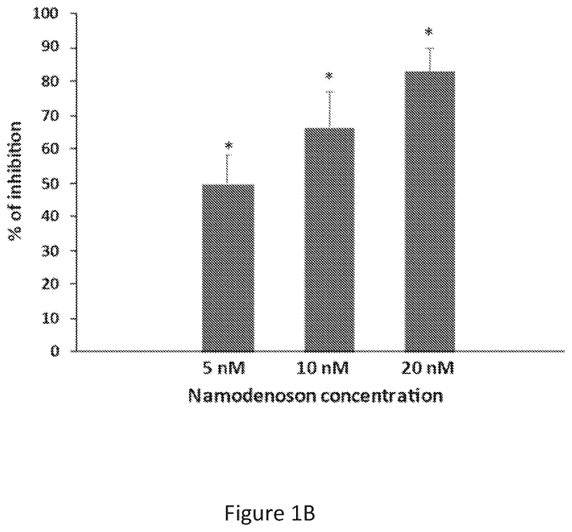

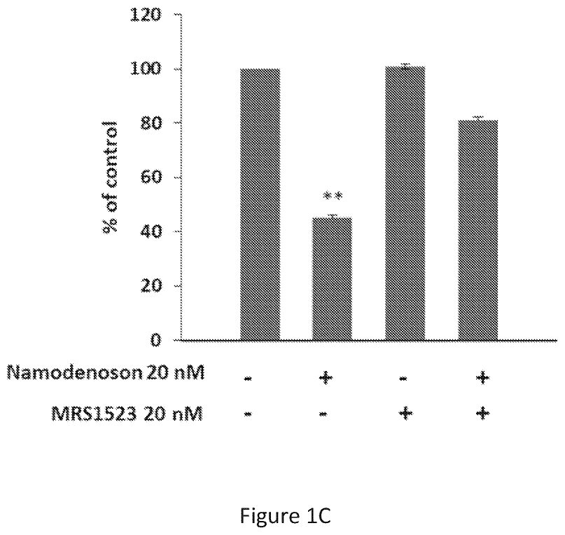

Namodenoson significantly inhibited the proliferation of the BxPC-3 cells in a dose dependent manner utilizing the 3H-thymidine incorporation assay (FIG. 1A).

In vitro analysis using the Presto Blue assay demonstrated a significant dose-dependent inhibition of BxPC-3 cell growth upon treatment with namodenoson (inhibition of 49.7%+8.2%, 66.3%±10.5% and 82.7%±7.1% for 5 nM, 10 nM, and 20 nM namodenoson, respectively, p<0.001 each; FIG. 1B).

Adding the A₃AR antagonist MRS1523 to the cells (with/without namodenoson) and assessing cell growth using the Presto Blue assay demonstrated that the namodenoson inhibitory effect was A₃AR-mediated, since treatment with 20 nM MRS1523 had no effect on cell growth (after incubation of 24 h, the growth was 100.8%+11.1% of the control), treatment with 20 nM namodenoson had an inhibitory effect (cell growth was 45.0%+4.2% of the control), and adding MRS1523 to namodenoson diminished the namodenoson inhibitory effect (cell growth was 81.1%+6.3% of the control) (FIG. 1C).

### Example 2: Namodenoson Inhibited Tumor Growth In Vivo

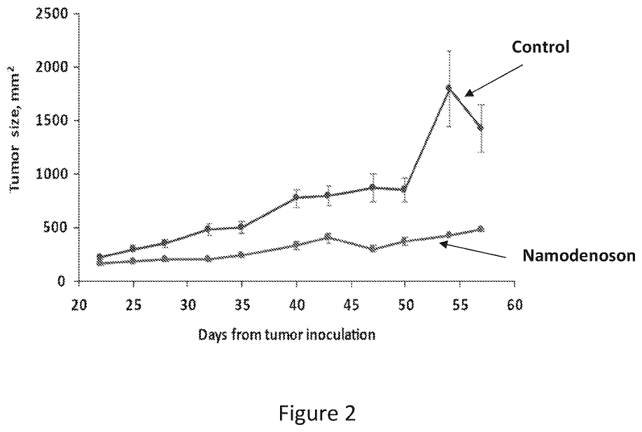

Analysis of the effect of namodenoson (10 μg/kg) given twice daily to nude mice inoculated with BxPC-3 cells for a total of 35 days (from Day 22 to Day 57 post tumor inoculation) demonstrated a significant inhibitory effect of namodenoson on tumor growth (inhibition of 67.7%+15.2% by Day 57 from tumor inoculation vs. control, p<0.05; FIG. 2).

### Example 3: In Vitro Effects of Namodenoson on Signal Transduction

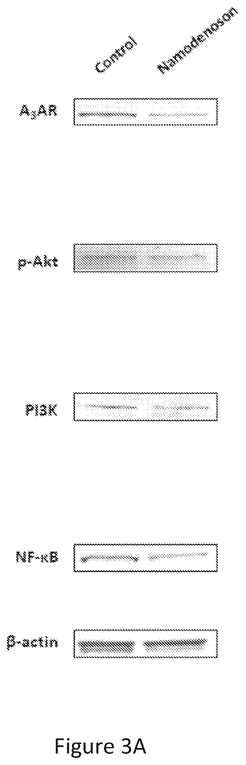

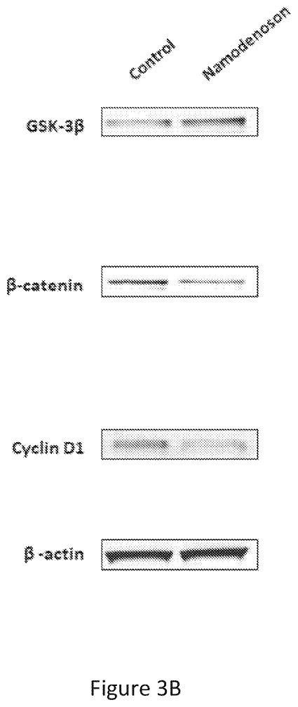

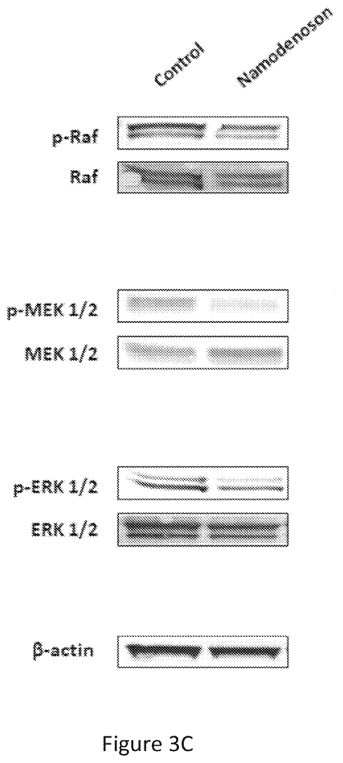

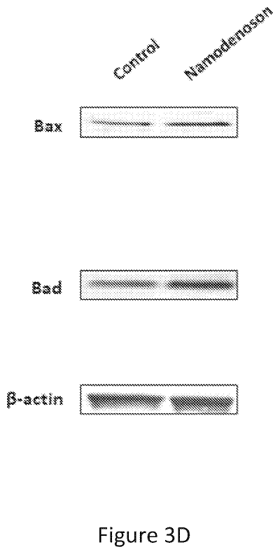

Western blot analyses using BxPC-3 cells showed statistically significant differences in the expression of regulatory proteins upon treatment with namodenoson. Specifically, namodenoson induced a decrease in A₃AR protein expression level, and the downstream regulatory proteins p-Akt, PI3K, and NF-κB were all downregulated (FIG. 3A) p<0.05. p values were measured by t-test vs. control. Moreover, the analysis of proteins within the Wnt signal transduction pathway revealed the upregulation of GSK-3β (p<0.05) and a decrease in the expression levels of β-catenin (p<0.05) and cyclin D1 (p<0.01) (FIG. 3B). A decrease in the expression levels of proteins downstream from the RAS signaling pathway (pRaf, pMEK 1/2, and pERK 1/2) was also observed (FIG. 3C) p<0.05. In addition, the two apoptotic proteins Bad and Bax were upregulated, suggesting that the apoptosis of the BxPC-3 cells was induced (FIG. 3D) p<0.05.

1-47. (canceled)

48. A method of treating pancreatic cancer, said method comprising administering to a mammalian subject in need thereof an A₃ adenosine receptor (A₃AR) ligand or a pharmaceutical composition comprising said A₃AR ligand, wherein said A₃AR ligand is 2-chloro-N⁶-(3-iodobenzyl)-adenosine-5′-N-methyluronamide (CI-IB-MECA, namodenoson).

49. The method of claim 48 wherein said pancreatic cancer is an advanced pancreatic cancer.

50. The method according to claim 48, wherein said method further comprises administration of an additional therapeutic agent, wherein said additional therapeutic agent is a chemotherapeutic agent selected from a group consisting of nucleoside chemotherapeutics, fluorouracil plus leucovorin, and fluorouracil plus leucovorin plus irinotecan plus oxaliplatin (known as FOLFIRINOX), or an immunotherapeutic agent, wherein said immunotherapeutic agent is an anti-tumor antibody and/or a checkpoint inhibitor.

51. The method according to claim 48, wherein said administration is performed prior to and/or after removal of the tumor.

52. The method according to claim 48 wherein said A₃AR ligand is administered once daily, twice daily, or thrice daily.

53. The method according to claim 48 wherein said A₃AR ligand is administered every 12 hours throughout the treatment period.

54. The method according to claim 53 wherein said treatment period is divided into cycles (e.g., 4-week cycles).

55. The method according to claim 48 wherein said A₃AR ligand is administered at an amount of 50 μg/kg-10 mg/kg body weight, preferably 100 μg/kg-5 mg/Kg body weight, or 200 μg/kg-1 mg/Kg body weight.

56. The method according to claim 48 wherein said CI-IB-MECA is administered orally in a dose of 1-50 mg, preferably 5-30 mg twice daily.

57. The method according to claim 48 wherein said subject receives the A₃AR ligand as a second-line therapy.

58. The method according to claim 48 wherein said administration is for a treatment period of at least 9 months, at least 10 months, at least one year, at least 2 years, at least 3 years, at least 4 years or at least 5 years.

59. A pharmaceutical composition comprising an A₃AR ligand, and a pharmaceutically acceptable carrier or diluent wherein said pharmaceutical composition is for treating a pancreatic cancer in a mammalian subject, wherein said A₃AR ligand is 2-chloro-N⁶-(3-iodobenzyl)-adenosine-5′-N-methyluronamide (CI-IB-MECA, namodenoson).

60. The pharmaceutical composition of claim 59 wherein said pancreatic cancer is an advanced pancreatic cancer.

61. The pharmaceutical composition according to claim 59 wherein said A₃AR ligand is administered once daily, twice daily, or thrice daily.

62. The pharmaceutical composition according to claim 59 wherein said A₃AR ligand is administered every 12 hours throughout the treatment period.

63. The pharmaceutical composition according to claim 59 wherein said treatment period is divided into cycles (e.g., 4-week cycles).

64. The pharmaceutical composition according to claim 59 wherein said A₃AR ligand is administered at an amount of 50 μg/kg-10 mg/kg body weight, preferably 100 μg/kg-5 mg/Kg body weight, or 200 μg/kg-1 mg/Kg body weight.

65. The pharmaceutical composition according to claim 59 wherein said CI-IB-MECA is administered orally in a dose of 1-50 mg, preferably 5-30 mg twice daily.

66. The pharmaceutical composition according to claim 59 wherein said subject received the A₃AR ligand as a second-line therapy.

67. The pharmaceutical composition according to claim 59 wherein said administration is for a treatment period of at least 9 months, at least 10 months, at least one year, at least 2 years, at least 3 years, at least 4 years or at least 5 years.
